(19) United States
(12) Patent Application Publication
(10) Pub. No.: US 2018/0304094 A1
(43) Pub. Date: Oct. 25, 2018

(54) SYSTEMS, DEVICES, AND METHODS FOR ADMINISTERING LOW-LEVEL LIGHT THERAPY

(71) Applicant: Vanderbilt University, Nashville, TN (US)

(72) Inventors: Adam Hicks, Franklin, TN (US); Brandon Fross, Ponca City, OK (US); John Mendoza, Nashville, TN (US); Laurel Piper, Western Springs, IL (US); Siegfried Schlunk, Brentwood, TN (US); Eliza Stedman, Charlotte, NC (US); Ahbid Zein-Sabatto, Nashville, TN (US)

(21) Appl. No.: 15/960,882

(22) Filed: Apr. 24, 2018

Related U.S. Application Data
(60) Provisional application No. 62/489,083, filed on Apr. 24, 2017.

(51) Int. Cl. A61N 5/06

(52) U.S. Cl. CPC A61N 5/0616; A61N 2005/0626; A61N 2005/0663; A61N 2005/0659; A61F 13/041; A61N 2005/0645; A61N 2005/063; A61N 2005/0668; A61N 2005/0652

(57) ABSTRACT

Described herein are systems, devices, and methods for administering low-level light therapy (LLLT). The systems, devices, and methods aim to accelerate wound healing and reduce the incidence of infection.

### CROSS-REFERENCE TO RELATED APPLICATIONS

This application claims the benefit of U.S. provisional patent application No. 62/489,083, filed on Apr. 24, 2017, and entitled “SYSTEMS, DEVICES, AND METHODS FOR ADMINISTERING LOW-LEVEL LIGHT THERAPY,” the disclosure of which is expressly incorporated herein by reference in its entirety.

### BACKGROUND

Diabetic foot ulcers are wounds, resistant to healing, usually on the bottom of the feet of diabetic patients. One fourth of the 29 million American diabetics will develop a diabetic foot ulcer at some point during their lifetime. The gold standard of care is an off-loading, total-contact cast that removes gait pressure from the wound. This treatment focuses on isolating these wounds in order to encourage passive healing over time. Furthermore, maintaining patient compliance is an ongoing difficulty with diabetic care. In light of delayed healing, diabetic patients with a non-healing ulcer are more prone to lower-extremity amputations.

### SUMMARY

Described herein are systems, devices, and methods for administering low-level light therapy (LLLT). The systems, devices, and methods aim to accelerate wound healing, reduce the bioburden on the wound site, and reduce the incidence of infection. For example, the systems, devices, and methods can have an anti-microbial effect in addition to promoting wound healing. As one example, a device can be used in the treatment of diabetic foot ulcers (DFUs). There is no current treatment option using LLLT that actively encourages diabetic foot ulcer healing, complements current procedures, and maintains patient compliance. Complications like infection often require the need for surgical intervention such as lower-extremity amputation. Previous studies have shown that exposing wounds to dose-specific levels of light can reduce wound size and promote healing. Incorporated into a standard of care (the total-contact orthopedic cast), the devices, systems, and methods described herein transfer light energy (e.g., using light-emitting diodes (LEDs)) from a power source to the wound site in order to introduce an active healing component for diabetic foot ulcers.

An example system for administering low-level light therapy to a patient in need thereof is described herein. The system can include a wound dressing configured to conform to a body part of the patient, where the wound dressing includes a cavity formed therein, the cavity being in proximity to a wound of the patient. The system can also include a light-emitting halo including one or more light sources, where the light-emitting halo is arranged within the cavity of the wound dressing to maintain wound offloading.

Additionally, the light-emitting halo can be spaced apart from the wound of the patient.

Alternatively or additionally, the light-emitting halo can be arranged in contact with at least a portion of the wound dressing.

Alternatively or additionally, a thickness of the light-emitting halo can be less than a thickness of the wound dressing.

Alternatively or additionally, the light-emitting halo can have a cylindrical shape with a hole in the center thereof.

Alternatively or additionally, the light-emitting halo can be formed from a biocompatible material or a biologically inert material.

Alternatively or additionally, the light-emitting halo can be formed from silicone.

Alternatively or additionally, the light-emitting halo can be formed from a waterproof material.

Alternatively or additionally, the system can further include a module box housing a power source and a control module. The module box can be operably coupled to the light-emitting halo and configured to control operation of the one or more light sources.

Alternatively or additionally, the system can further include a cable for operably coupling the module box and the light-emitting halo.

Alternatively or additionally, the light-emitting halo and the cable can be disposable.

Alternatively or additionally, the light-emitting halo and the cable can be sterile.

Alternatively or additionally, the one or more light sources can be a monochromatic light source.

Alternatively or additionally, the one or more light sources can be a source of blue light. For example, the source of blue light can have a wavelength of from about 450 nm to about 495 nm.

Alternatively or additionally, the one or more light sources can be a source of infrared light. For example, the source of infrared light can have a wavelength of from about 700 nm to about 1 mm.

Alternatively or additionally, the one or more light sources can be a source of blue light and a source of infrared light.

Alternatively or additionally, the one or more light sources can be one or more light emitting diodes.

Alternatively or additionally, the one or more light sources can be configured to emit light of at least 20 mW/cm².

Alternatively or additionally, the wound dressing can be a cast.

Alternatively or additionally, the body part of the patient can be a foot, and the wound can be located on a sole of the foot.

An example device for administering low-level light therapy to a patient in need thereof is also described herein. The device can include a module box housing a power source and a control module, a light-emitting halo including one or more light sources, and a cable for operably coupling the module box and the light-emitting halo. The light-emitting halo can be configured to removably couple a wound dressing on the patient.

An example method of treating a wound on a patient is also described herein. The method can include applying to the patient the system or device for administering LLLT as described herein, and illuminating the wound with a therapeutically effective amount of light from the one or more light sources of the light-emitting halo.

The systems, devices, and methods described herein can be automated, preprogrammed for active healing, and integrated into a total-contact off-loading cast. The device is weatherproof, low-risk, and has a low-profile. The device can include: a module box (sometimes referred to herein as a “module unit”), a light-emitting halo, and a connector unit. The module box, secured to the cast (and in some implementations externally secured to the cast), controls the dose-specific levels of light emission, which is transmitted via the light-delivering halo. These two pieces are connected via a 3D-printed connector piece which is secured to the module unit casing.

As noted above, devices for administering low-level light therapy (LLLT) to a wound site are provided herein. The devices can include a module box and a halo comprising one or more light sources (e.g., one or more light-emitting diodes configured to emit the desired wavelength of light (e.g., blue light and/or infrared light) for a therapeutic application). A cable can be used to electrically connect the module box and the halo to supply electrical power and/or control signals to the one or more light sources. Optionally, in an alternative implementation, one or more light emitters can be housed in the module box and embedded fiber optics can be used to transfer light from the light source within the module box to the wound site via the halo. The module box can house a power source (e.g., batteries) and a control module configured to control the delivery of light to a patient. In some embodiments, the module box can further include a heat sink, such as a thermal pad, to absorb heat produced by the components in the module box. Additional auxiliary components found in the module box can optionally include an interactive liquid crystal display (LCD) screen that displays the device status (e.g. battery level, treatment dosage, etc.), overheating shutdown override circuitry, and desiccants to eliminate moisture accumulation within the device. The module box can be configured to securely attach to the exterior of a cast or other wound dressing. As discussed above, the halo can include one or more light sources to transfer light to the wound site. The halo can be custom-molded to conform to the patient's anatomy. In some embodiments, the halo can be formed from a compliant, biocompatible material, such as silicone. The device can be designed to be weatherproof (e.g., waterproof or water-resistant), shock-resistant, heat resistant, or a combination thereof.

The device can be used to actively encourage wound healing. For example, the device can be used to treat wounds associated with diabetes, such as diabetic foot ulcers. Diabetic foot ulcers are a significant clinical problem. In particular, diabetic foot ulcers are susceptible to infection. Negative wound progression is an indication of improper treatment and often introduces the need for surgical intervention, such as lower-extremity amputation. The automated delivery of therapeutic light can be used to help reduce the risk of negative wound progressions and accelerate the healing of diabetic foot ulcers. These devices can be adopted in a clinical setting to treat patients with severe and non-severe foot ulcers. These devices can be used with minimal risk to the patient, and can complement current standards of care for wound management, including current standards of care for the management of diabetic foot ulcers.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a diagram illustrating an example low-level light therapy system according to implementations described herein.

FIG. 2 is a diagram illustrating an example low-level light therapy device according to implementations described herein.

FIG. 3 is a cross-sectional view illustrating the light-emitting halo and wound dressing according to implementations described herein.

FIG. 4 is a diagram illustrating a perspective view of the light-emitting halo according to implementations described herein.

FIG. 5 is a diagram illustrating a top view of the light-emitting halo according to implementations described herein.

FIG. 6 is a diagram illustrating the module box according to implementations described herein.

FIG. 7 is a diagram illustrating a connector for the module box according to implementations described herein.

FIG. 8 is a diagram illustrating the cable and light-emitting halo according to implementations described herein.

FIG. 9 is an example computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for using an LLLT device/system to treat diabetic foot ulcers, it will become evident to those skilled in the art that the implementations are not limited thereto, but are applicable for treating other types of wounds including burns, normal ulcers, and surgical wounds.

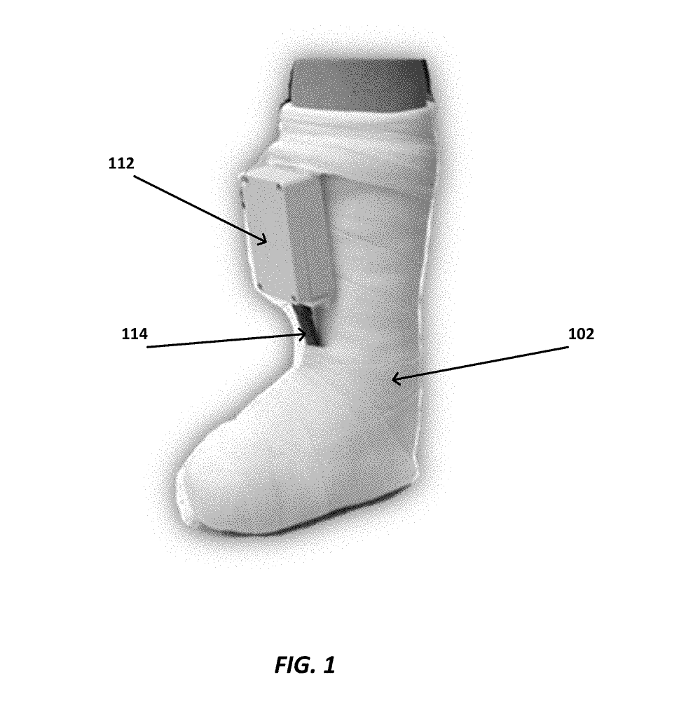

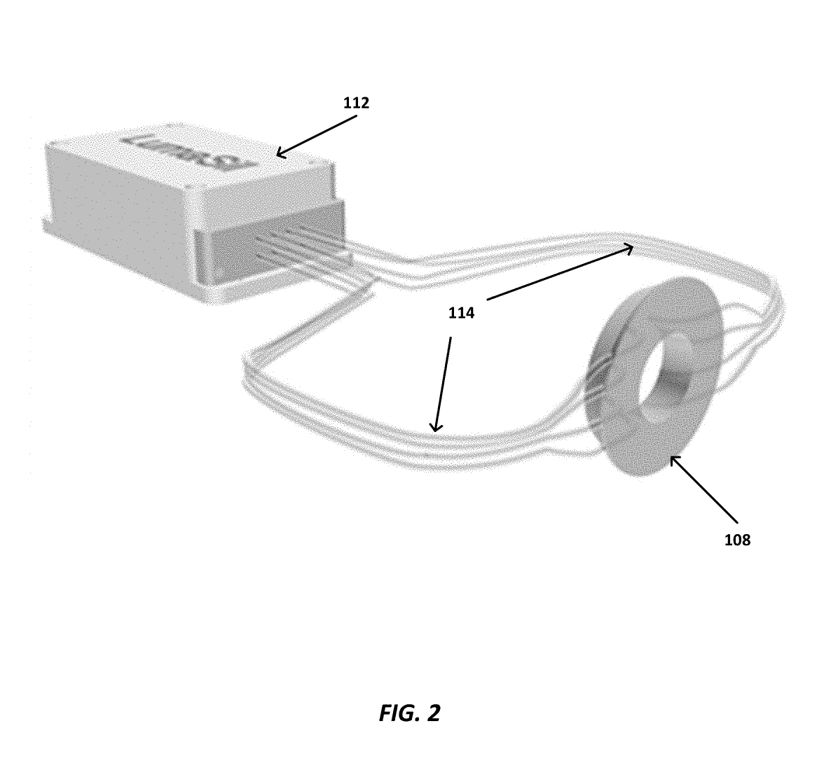

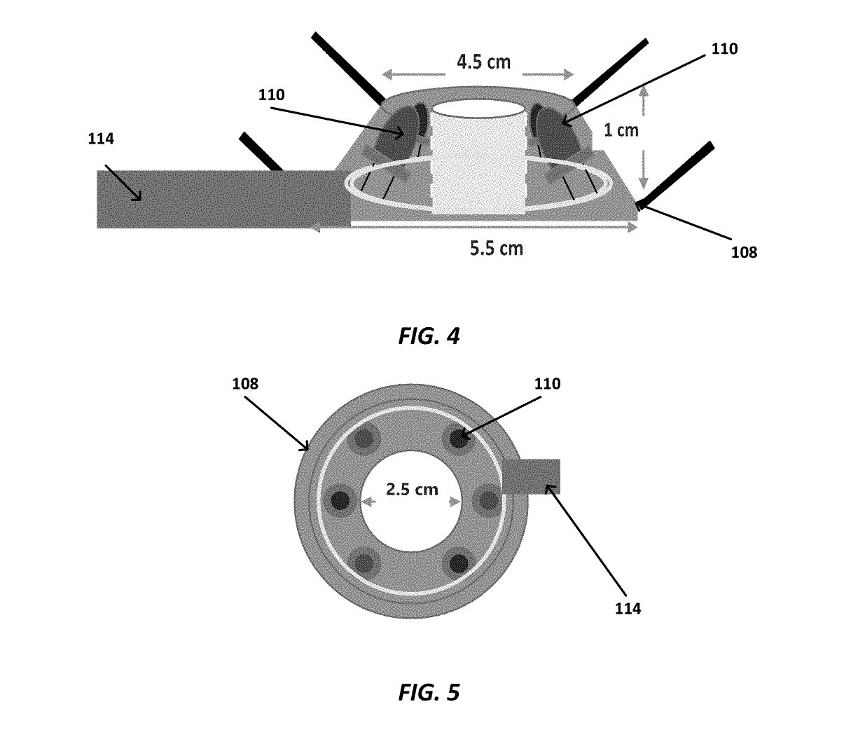

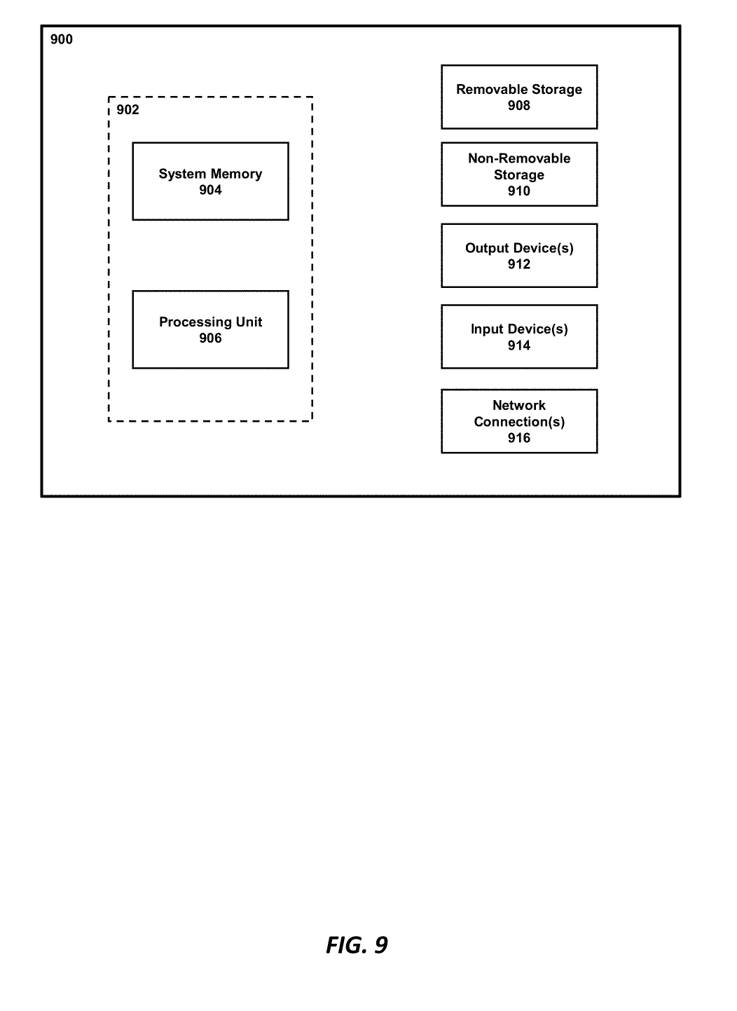

Referring now to FIGS. 1-5, an example low-level light therapy system is shown. The system can include a low-level light therapy device and a wound dressing 102. The low-level light therapy device can include a module box 112 (or “module unit”), a light-emitting halo 108 including one or more light sources 110, and a cable 114 for operably coupling the module box 112 and the light-emitting halo 108. In some implementations, the one or more light sources 110 can be embedded in the light-emitting halo 108. In some implementations, the one or more light sources 110 can be attached or secured to the light-emitting halo 108. The module box 112 can house a power source and a control module (or “control unit”) (e.g., a computing device such as the computing device 900 of FIG. 9). The module box 112 can be durable, waterproof, and shockproof. Optionally, the module box 112 can be made of polycarbonate material. This disclosure contemplates that the module box 112 can be any shape and/or size. The module box 112 can be operably coupled to the light-emitting halo 108 (e.g., using the cable 114), and the module box 112 can be configured to control operation of the one or more light sources 110. For example, the module box 112 can include a control module (e.g., a microprocessor), which can be configured to control operation of the light sources 110. For example, the module box 112 can include computer-executable instructions stored in memory (e.g., software) that, when executed by the control module, control operation of the light sources. Optionally, the module box 112 can be programmed to deliver light via the light-emitting halo 108 at predetermined times (e.g., daily) and/or for predetermined durations (e.g., 20 minutes), etc. In other words, the module box 112 can be programmed with an LLLT treatment regimen. The light sources 110 can be controlled to deliver light at a predetermined intensity for a predetermined period of time. In some implementations, the light sources 110 can be optionally be controlled to emit light of at least 20 mW/cm². In some implementations, the light sources 110 can be optionally be controlled to emit light of up to 50 mW/cm². In some implementations, the light sources 110 can optionally be controlled to emit light of at about 25 mW/cm² for about 20 minutes a day. Daily treatment can be provided until the patient's wound heals. In some implementations, treatment is provided for a total of about 2-4 weeks. It should be understood that the intensity of light emission and/or daily delivery time and/or delivery pattern and/or treatment duration are provided only as an example. This disclosure contemplates using other light emission intensities and/or daily delivery times and/or delivery pattern and/or treatment durations. This disclosure also contemplates that the intensity of light emission and/or delivery time and/or delivery pattern and/or treatment duration can be varied to achieve therapeutic effect (e.g., higher light emission intensity applied for a shorter period of time/lower light emission intensity applied for a longer period of time). The light-emitting halo 108 can include a plurality of light sources 110 (e.g., six LEDs as shown in FIG. 5). The light sources 110 can be arranged in a spaced apart manner within the light-emitting halo 108. Optionally, the light sources 110 can be arranged at an angle with respect to the wound 106 (e.g., as shown in FIG. 4), e.g., in order to increase therapeutic effect. It should be understood that the number and/or arrangement of the light sources 110 embedded in the light-emitting halo 108 are provided only as examples. Optionally, the light sources 110 can be secured in and/or attached to the light-emitting halo 108 using biocompatible adhesives. The light-emitting halo 108 can be configured to removably couple with the wound dressing 102. Additionally, the light-emitting halo 108 can be arranged within a cavity 104 of the wound dressing 102 to maintain wound offloading.

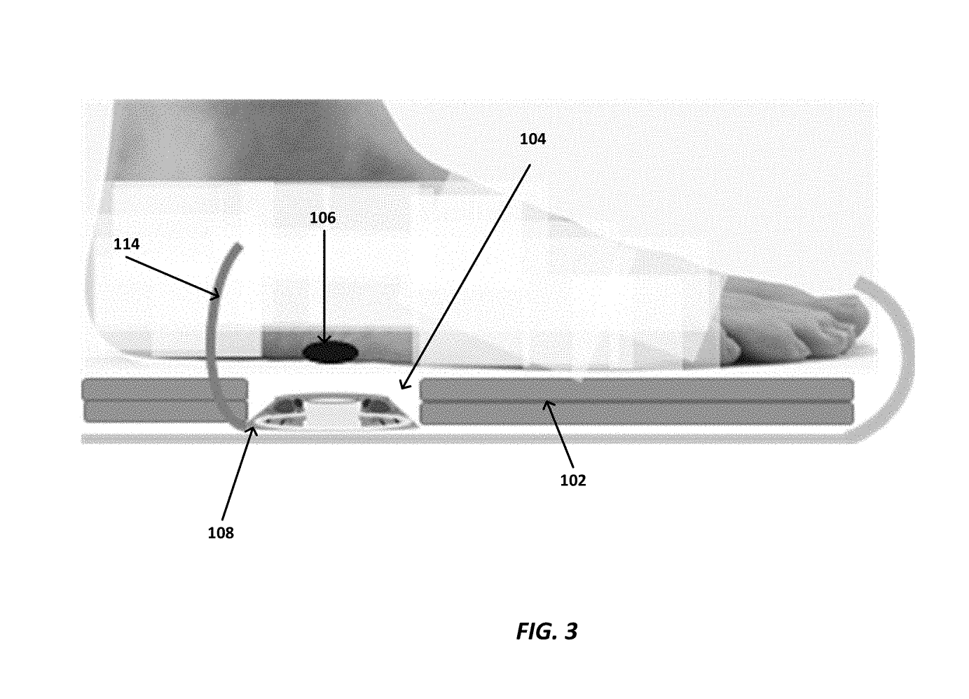

The wound dressing 102 can be configured to conform to a body part of the patient. In some implementations, the wound dressing 102 can be a cast, for example. The wound dressing 102 can have the cavity 104 formed therein, for example, in proximity to the wound 106. This is shown in FIG. 3, where the wound dressing 102 is a cast on the patient's foot, and the wound 106 is on the sole of the patient's foot. In particular, the patient's foot is wrapped with cotton wrapping with the exception of near the wound 106. A rigid material such as fiberglass is provided over the patient's foot, which is wrapped in cotton. One or more layers of flexible material (e.g., felt) can optionally be arranged between the cast and the patient's cotton-wrapped foot. The wound 106 can be exposed through the cavity 104 in the wound dressing 102 as shown in FIG. 3 to maintain offloading. The cavity 104 can be formed in one or more of the layers of the wound dressing 102 in order to expose the wound 106. This disclosure contemplates that the size and/or shape of the cavity 104 can be based on the size and/or shape of the wound 106. It should be understood that the wound dressing type, wound, and/or wound locations are provided only as examples.

As shown in FIG. 3, the light-emitting halo 108 can be spaced apart from the wound 106 of the patient. In other words, the light-emitting halo 108 does not make contact with the wound 106. For example, the light-emitting halo 108 can be in close proximity (e.g., 2-3 cm) but not in contact with the wound 106. The light-emitting halo 108 is therefore incorporated into the wound dressing 102 such that light shines on the wound 106. Optionally, the light-emitting halo 108 can be arranged in contact with at least a portion of the wound dressing 102, which prevents the light-emitting halo 108 from making contact with the wound 106. Optionally, a thickness of the light-emitting halo 108 is less than a thickness of the wound dressing 102.

Referring again to FIGS. 1-5, the light-emitting halo 108 can have a cylindrical shape with a hole in the center thereof. This shape can fit within the cavity 104 of the wound dressing 102 (e.g., within both the cotton layer directly around the foot and the felt layers between the foot and rigid material) as shown in FIG. 3. This disclosure contemplates that the hole in the center of the light-emitting halo 108 ensures that no material interferes with the wound 106 to ensure offloading and/or absorbent material such as gauze can be placed within the hole without obstructing the light emitted from the light sources 110 embedded in the light-emitting halo 108. It should be understood that the light-emitting halo 108 can have shapes and/or sizes other than those shown in the figures, which are provided only as examples. Example dimensions for the light-emitting halo 108 are shown in FIGS. 4 and 5. For example, the light-emitting halo 108 can have a bottom-side outer diameter of about 5.5 cm, a top-side outer diameter of about 4.5 cm, and an inner hole diameter of about 2.5 cm. It should be understood that the light-emitting halo 108 can have dimensions other than those shown in the figures, which are provided only as examples. Optionally, the light-emitting halo 108 is formed from a biocompatible material. Alternatively or additionally, the light-emitting halo 108 can optionally be formed from a biologically inert material. Alternatively or additionally, the light-emitting halo 108 can optionally be formed from silicone. Alternatively or additionally, the light-emitting halo 108 can optionally be formed from a waterproof material. The material for the light-emitting halo 108 can be selected such that it absorbs compressive force and prevents damage to the embedded light sources 110 (e.g., LEDs).

This disclosure contemplates that the light-emitting halo 108 and/or cable 114 can experience physical wear during use. In some implementations, the light-emitting halo 108 and the cable 114 are disposable. For example, the light-emitting halo 108 and/or cable 114 can be designed for single use. Optionally, the light-emitting halo 108 and the cable 114 can be single-use and optionally sterile. In other words, the module box 112 can be non-disposable, and the light-emitting halo 108/cable 114 can be disposable (and optionally sterile). In this way, the light-emitting halo 108/cable 114 can be periodically replaced (e.g., weekly) during wound 106 debridement treatment, and the light-emitting halo 108/cable 114 would not need to be sterilized. The light-emitting halo 108/cable 114 can be detached from the module box 112 (e.g., by unplugging the cable 114), and a new light-emitting halo 108/cable 114 can be provided. This disclosure also contemplates that this can reduce the time needed for periodic treatment. For example, rather than removing the light-emitting halo 108/cable 114 and performing in depth sterilization and subsequent replacement, these components can be detachable from the module box 112 and then disposed.

In some implementations, the one or more light sources 110 comprise a monochromatic light source. For example, the light sources 110 can be a source of blue light such as blue light having a wavelength of from about 450 nm to about 495 nm. Optionally, the blue light has a wavelength of about 470 nm. A light-emitting diode (LED) can be a source of blue light. For example, a T-1¾ (5 mm×5 mm) blue LED from BROADCOM INC. of San Jose, Calif. having a wavelength of 470 nm and intensity of 1200 millicandela (mcd) can optionally be used. Alternatively or additionally, the light sources 110 can be a source of infrared light such as infrared light having a wavelength from about 700 nm to about 1 mm. Optionally, the infrared light has a wavelength from about 840 nm to about 900 nm. Optionally, the infrared light has a wavelength of about 890 nm. An LED can be a source of infrared light. For example, a 8.7 mm×5.8 mm×5.8 mm infrared LED from VISHAY INTERTECHNOLOGY, INC. of Malvern, Pa. having a wavelength of 890 nm and intensity of 1400 mW/sr can optionally be used. Optionally, the light sources 110 can be a source of blue light and a source of infrared light. As described above, the light sources 110 can optionally be controlled to emit light of at about mW/cm² for about 20 minutes daily. Optionally, in some implementations, the light sources 110 can be light-emitting diodes (LEDs). It should be understood that the LEDs provided above are examples only and that other LEDs can be used.

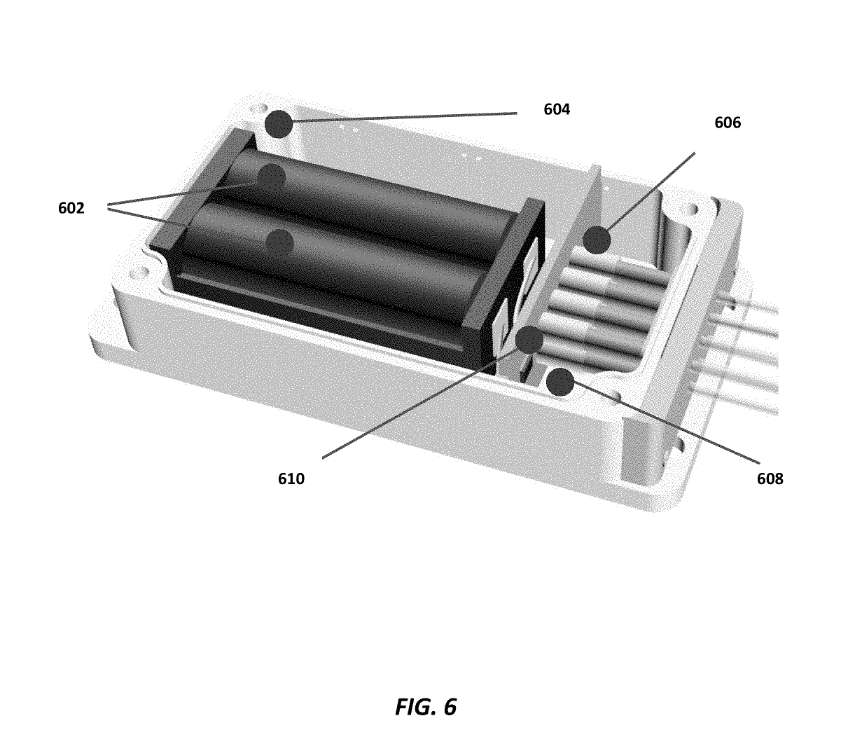

Referring now to FIG. 6, a diagram illustrating an example module box (e.g., the module box 112 of FIG. 1) is shown. The module box can include rechargeable batteries (e.g., lithium-ion batteries) 602, a water-resistant polycarbonate box 604, a microcontroller for controlling light delivery 606, and a heat sink (e.g., graphite sheet or other thermal pad) 608 to distribute excess heat. The light sources (e.g., light sources 110 of FIG. 4) produce heat during normal operations, and the heat sink 608 can be used to dissipate some of this heat throughout the module box. Optionally, in some implementations (e.g., when the light sources are not embedded in the light-emitting halo as described above), the module box can include one or more light sources 610 (e.g., blue and infrared LEDs emit light to provide wound healing and antimicrobial effects). Light can be delivered to the patient's wound using fiber optic cables.

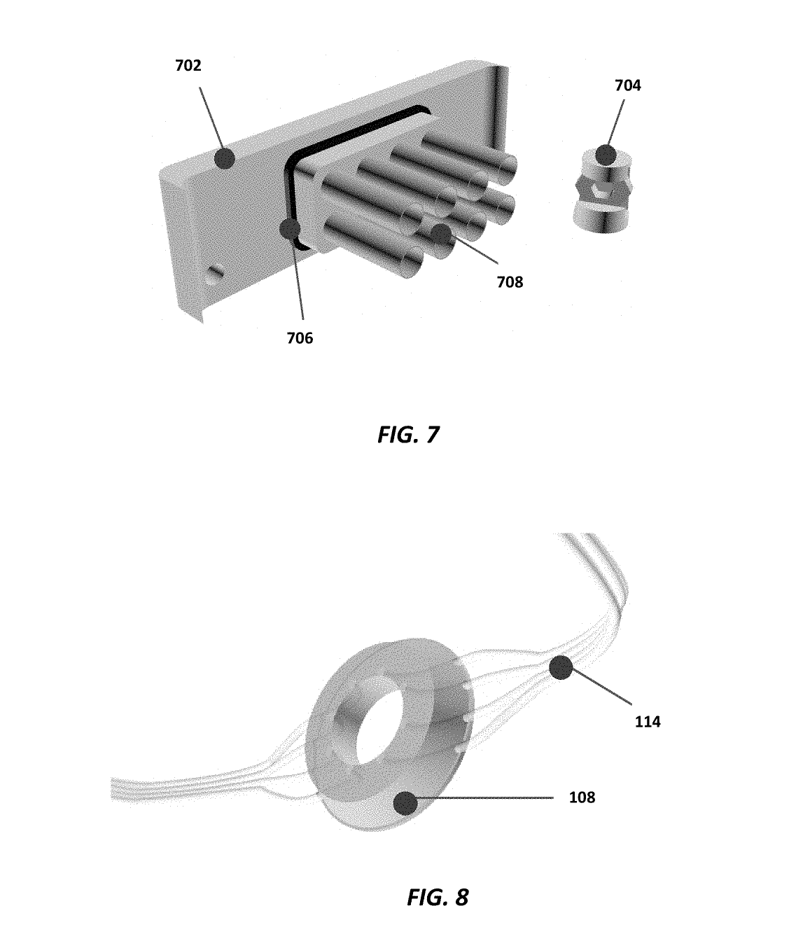

Referring now to FIG. 7, a diagram illustrating a connector for the module box (e.g., the module box 112 of FIG. 1) is shown. The connector can include a connector piece 702 that allows for connection between the module box and cable, a bolt enclosure 704 that secures the connector piece 702 to the module box, and a seal 706 (e.g., rubber O-ring) to maintain water-resistance. Optionally, the connector piece 702 and/or the bolt enclosure 704 can be produced using a three-dimensional (3D) printer. It should be understood that the connector piece 702 and/or the bolt enclosure 704 can be produced using other manufacturing methods. Optionally, in some implementations (e.g., when the light sources are not embedded in the light-emitting halo as described above), the 3D-printed connector piece 702 allows for fiber optic-LED attachment using copper tubing 708 for aligning fiber optic cables with the LEDs.

Referring now to FIG. 8, a diagram illustrating the cable 114 and the light-emitting halo 108 are shown. This disclosure contemplates that the cable 114 can be a wired, wireless, and/or optical link that facilitates energy and/or data exchange between the light-emitting halo 108 and the module box.

It should be appreciated that the logical operations described herein with respect to the various figures may be implemented (1) as a sequence of computer implemented acts or program modules (i.e., software) running on a computing device (e.g., the computing device described in FIG. 9), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 9, an example computing device 900 upon which embodiments of the invention may be implemented is illustrated. It should be understood that the example computing device 900 is only one example of a suitable computing environment upon which embodiments of the invention may be implemented. Optionally, the computing device 900 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 900 typically includes at least one processing unit 906 and system memory 904. Depending on the exact configuration and type of computing device, system memory 904 may be volatile (such as random access memory (RAM)), non-volatile (such as read-only memory (ROM), flash memory, etc.), or some combination of the two. This most basic configuration is illustrated in FIG. 9 by dashed line 902. The processing unit 906 may be a standard programmable processor that performs arithmetic and logic operations necessary for operation of the computing device 900. The computing device 900 may also include a bus or other communication mechanism for communicating information among various components of the computing device 900.

Computing device 900 may have additional features/functionality. For example, computing device 900 may include additional storage such as removable storage 908 and non-removable storage 910 including, but not limited to, magnetic or optical disks or tapes. Computing device 900 may also contain network connection(s) 916 that allow the device to communicate with other devices. Computing device 900 may also have input device(s) 914 such as a keyboard, mouse, touch screen, etc. Output device(s) 912 such as a display, speakers, printer, etc. may also be included. The additional devices may be connected to the bus in order to facilitate communication of data among the components of the computing device 900. All these devices are well known in the art and need not be discussed at length here.

The processing unit 906 may be configured to execute program code encoded in tangible, computer-readable media. Tangible, computer-readable media refers to any media that is capable of providing data that causes the computing device 900 (i.e., a machine) to operate in a particular fashion. Various computer-readable media may be utilized to provide instructions to the processing unit 906 for execution. Example tangible, computer-readable media may include, but is not limited to, volatile media, non-volatile media, removable media and non-removable media implemented in any method or technology for storage of information such as computer readable instructions, data structures, program modules or other data. System memory 904, removable storage 908, and non-removable storage 910 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 906 may execute program code stored in the system memory 904. For example, the bus may carry data to the system memory 904, from which the processing unit 906 receives and executes instructions. The data received by the system memory 904 may optionally be stored on the removable storage 908 or the non-removable storage 910 before or after execution by the processing unit 906.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A system for administering low-level light therapy to a patient in need thereof, the system comprising:
a wound dressing configured to conform to a body part of the patient, wherein the wound dressing comprises a cavity formed therein, the cavity being in proximity to a wound of the patient; and
a light-emitting halo comprising one or more light sources, wherein the light-emitting halo is arranged within the cavity of the wound dressing to maintain wound offloading.

2. The system of claim 1, wherein the light-emitting halo is spaced apart from the wound of the patient.

3. The system of claim 1, wherein the light-emitting halo is arranged in contact with at least a portion of the wound dressing.

4. The system of claim 1, wherein a thickness of the light-emitting halo is less than a thickness of the wound dressing.

5. The system of claim 1, wherein the light-emitting halo has a cylindrical shape with a hole in the center thereof.

6. The system of claim 1, wherein the light-emitting halo is formed from a biocompatible material or a biologically inert material.

7. (canceled)

8. The system of claim 1, wherein the light-emitting halo is formed from a waterproof material.

9. The system of claim 1, further comprising a module box housing a power source and a control module, wherein the module box is operably coupled to the light-emitting halo and configured to control operation of the one or more light sources.

10. The system of claim 9, further comprising a cable for operably coupling the module box and the light-emitting halo.

11. The system of claim 10, wherein the light-emitting halo and the cable are disposable.

12. The system of claim 10, wherein the light-emitting halo and the cable are sterile.

13. The system of claim 1, wherein the one or more light sources comprise a monochromatic light source.

14. The system of claim 1, wherein the one or more light sources comprise a source of blue light.

15. The system of claim 14, wherein the source of blue light has a wavelength of from about 450 nm to about 495 nm.

16. The system of claim 1, wherein the one or more light sources comprise a source of infrared light.

17. The system of claim 16, wherein the source of infrared light has a wavelength of from about 700 nm to about 1 mm.

18. The system of claim 1, wherein the one or more light sources comprise a source of blue light and a source of infrared light.

19. The system of claim 1, wherein the one or more light sources comprise one or more light emitting diodes.

20. The system of claim 1, wherein the one or more light sources are configured to emit light of at least 20 mW/cm².

21. (canceled)

22. The system of claim 1, wherein the body part of the patient is a foot, and wherein the wound is located on a sole of the foot.

23-42. (canceled)
